(19) United States
(12) Patent Application Publication
(10) Pub. No.: US 2024/0105305 A1
(43) Pub. Date: Mar. 28, 2024

(54) RADIATION DOSE MANAGEMENT SYSTEM, RADIATION DOSE MANAGEMENT DEVICE, RADIATION DOSE MANAGEMENT METHOD, AND RECORDING MEDIUM

(71) Applicant: Konica Minolta, Inc., Tokyo (JP)

(72) Inventor: Yuki Tomiyama, Tokyo (JP)

(73) Assignee: Konica Minolta, Inc., Tokyo (JP)

(21) Appl. No.: 18/372,829

(22) Filed: Sep. 26, 2023

(30) Foreign Application Priority Data
Sep. 28, 2022 (JP) 2022-154615

(51) Int. Cl. G16H 20/40

(52) U.S. Cl. CPC G16H 20/40

(57) ABSTRACT

A radiation dose management system including a hardware processor that: automatically acquires, from a storage, predetermined information related to an examination of nuclear medicine, and automatically associates the predetermined information with information on a diagnostic reference level corresponding to the examination.

### CROSS-REFERENCE TO RELATED APPLICATIONS

The entire disclosure of Japanese Patent Application No. 2022-154615, filed on Sep. 28, 2022, including description, claims, drawings and abstract is incorporated herein by reference.

### BACKGROUND

### Technical Field

The present invention relates to a radiation dose management system, a radiation dose management device, a radiation dose management method, and a recording medium.

### Description of Related Art

In the medical field, an examination device such as a single photon emission computed tomography (SPECT) device or a positron emission tomography (PET) device that performs a nuclear medicine examination is used. The examinations where SPECT devices, PET devices, and the like are utilized, a radiopharmaceutical is administered to a patient. Next, the examination device performs imaging by detecting gamma rays emitted from the radioisotope included in the radiopharmaceutical. It is important to appropriately manage the exposure radiation dose of the patient by the gamma rays.

For example, Japanese Unexamined Patent Publication No. 2020-035412 describes the following technology for exposure radiation dose management.

The exposure radiation dose management device acquires information on the type of radiopharmaceutical, administration time, administered amount, examination method, and the like in a nuclear medicine examination. Next, the exposure radiation dose management device calculates an exposure radiation dose of the patient based on the acquired information.

In the examination of nuclear medicine, there are a case where a radiopharmaceutical is administered twice in one examination, a case where a radiopharmaceutical is administered only once in one examination, and the like. In a case where a radiopharmaceutical is administered twice in one examination, the administered amount is generally smaller than in a case where a radiopharmaceutical is administered once in each of two examinations. That is, the reference value for the administered amount of the radiopharmaceutical when the radiopharmaceutical is administered twice in one examination is less than the sum of the reference values when the radiopharmaceutical is administered once each in two examinations.

For example, a case where the excessive determination of the administered amount of the radiopharmaceutical is performed with reference to the sum of the reference values in a case where the radiopharmaceutical is administered once in each of two examinations although the radiopharmaceutical is administered twice in one examination will be described. In this case, a larger reference value than the original reference value is referred to, and thus there is a risk that the user is not aware of the excessive administration. When the radiopharmaceutical is excessively administered, the exposure radiation dose of the patient also becomes excessive.

Japanese Unexamined Patent Publication No. 2020-035412 does not describe a reference value related to the administered amount of the radiopharmaceutical corresponding to the examination to be performed, and cannot deal with the above issues.

### SUMMARY

One or more embodiments of the present invention provide a radiation dose management system, a radiation dose management device, a radiation dose management method, and a recording medium that can more appropriately manage an exposure radiation dose of a patient in a nuclear medicine examination.

According to an aspect of the present invention, a radiation dose management system includes: a hardware processor that automatically acquires, from a storage, predetermined information related to an examination of nuclear medicine, wherein the hardware processor automatically associates the predetermined information with information on a diagnostic reference level corresponding to the examination.

According to an aspect of the present invention, a radiation dose management device includes: a hardware processor that automatically acquires, from a storage, predetermined information related to an examination of nuclear medicine, wherein the hardware processor automatically associates the predetermined information with information on a diagnostic reference level corresponding to the examination.

According to an aspect of the present invention, a radiation dose management method includes: examination information acquiring that is automatically acquiring, from a storage, predetermined information related to an examination of nuclear medicine; and associating that is automatically associating the predetermined information with information on a diagnostic reference level corresponding to the examination.

According to an aspect of the present invention. a non-transitory recording medium stores a computer readable program causing a computer included in a radiation dose management system to function as a hardware processor that automatically acquires, from a storage, predetermined information related to an examination of nuclear medicine, wherein the hardware processor automatically associates the predetermined information with information on a diagnostic reference level corresponding to the examination.

### BRIEF DESCRIPTION OF THE DRAWINGS

The advantages and features provided by one or more embodiments of the invention will become more fully understood from the detailed description given hereinafter and the appended drawings which are given by way of illustration only, and thus are not intended as a definition of the limits of the present invention, and wherein:

FIG. 1 is a diagram of a system configuration of a radiation dose management system;

FIG. 2 is a diagram illustrating an example of an examination name in examination order information;

FIG. 3 is a block diagram illustrating a functional configuration of a radiation dose management device;

FIG. 4 is a table illustrating an example of information stored in a diagnostic reference level database;

FIG. 5 is a flowchart illustrating radiation dose management processing executed in the radiation dose management device of a first embodiment;

FIG. 6 is a diagram showing an example of displaying the reference value of the diagnostic reference level, the total of the actual administered amount, and the determination result; and

FIG. 7 is a flowchart illustrating radiation dose management processing executed in a radiation dose management device of a second embodiment.

### DETAILED DESCRIPTION

Hereinafter, embodiments of a radiation dose management system according to the present invention will be described. However, the present invention is not limited to the examples illustrated in the drawings.

### First Embodiment

### (1 Configuration of Radiation Dose Management System)

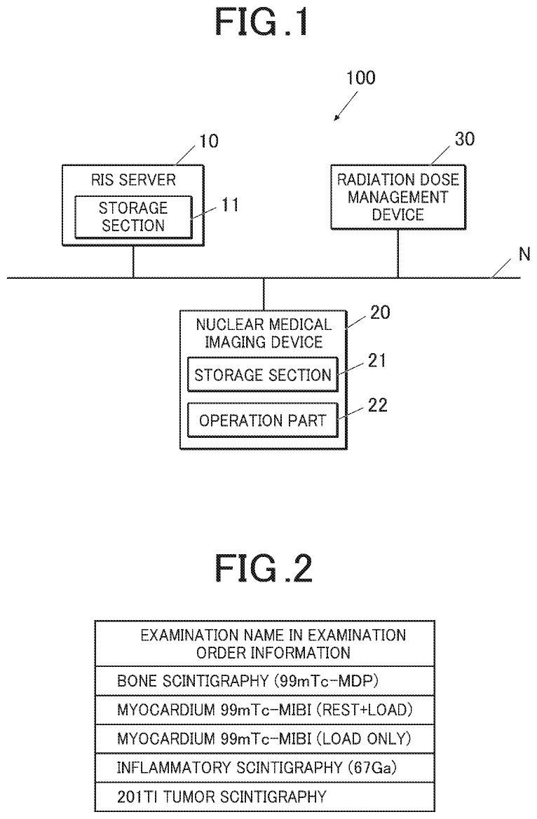

FIG. 1 illustrates a system configuration example of a radiation dose management system 100.

As illustrated in FIG. 1, the radiation dose management system 100 includes a radiology information system (RIS) server 10 serving as a radiation information device, a nuclear medical imaging device 20, and a radiation dose management device 30.

The respective devices included in the radiation dose management system 100 are connected to each other via a communication network N such as a local area network (LAN) or a wide area network (WAN) so as to transmit and receive data. The devices included in the radiation dose management system 100 conform to the HL7 (Health Level Seven) standard or the DICOM (Digital Image and Communications in Medicine) standard. Communication between the devices included in the radiation dose management system 100 is performed according to HL7 or DICOM.

The RIS server 10 manages information in the radiology department, such as appointments for examinations and treatments performed by the radiation device and examination results.

The RIS server 10 includes a storage section (storage) 11 and manages the examination order information stored in the storage section 11. The RIS server 10 transmits the examination order information to the modality (such as the nuclear medical imaging device 20) to be examined.

The examination order information includes patient information, examination information, and the like.

The patient information is information on a patient (subject). The patient information includes a patient ID, a patient name, date of birth, age, sex, height, weight, and the like.

The examination information is information on an examination. The examination information includes an examination ID, an examination date and time, a modality (a SPECT device, a PET device, or the like), an examination site, an examination purpose, an examination description, an examination name, and the like.

The predetermined information related to the examination of the nuclear medicine is an examination name, information of examinations included in one examination, and the like. The predetermined information on a examination of nuclear medicine is not limited to this as long as it includes information on an examination site in the nuclear medical examination, a type of a radiopharmaceutical (e.g., a name of the radiopharmaceutical) to be administered to a patient in the examination, and the number of times of administration of the radiopharmaceutical in one examination.

In the present embodiment, the predetermined information related to the nuclear medicine examination is an examination name.

FIG. 2 illustrates an example of an examination name in the examination order information according to the present embodiment.

As shown in FIG. 2, the examination name includes information on the examination site in the nuclear medical examination, the type of the radiopharmaceutical (for example, the name of the radiopharmaceutical) administered to the patient in the examination, and the number of times of administration.

The examination site is, for example, a bone, a cardiac muscle (blood flow), inflammation, or a tumor.

The radiopharmaceutical names are, for example, 99 mTc-MDP, 99 mTc-MIBI, 67Ga, 201T1, and the like.

As the number of times of administration, for example;(rest+load) indicates two times of administration; and (load only) and no description for rest or load indicate one time of administration.

The nuclear medical imaging device 20 is, for example, a SPECT device, a PET device, or the like. The nuclear medical imaging device 20 generates a radiation image by imaging radiation emitted to the outside of the body when a radioisotope (RI) is inhaled or intravenously injected into the body of a patient.

Operations of the nuclear medical imaging device 20 will be described.

The nuclear medical imaging device 20 receives the examination order information from the RIS server 10.

Next, the nuclear medical imaging device 20 receives, through an operation part (user interface) 22, input of an administration time and a administered amount of a radiopharmaceutical administered to a patient in a nuclear medical examination, a type of the radiopharmaceutical (e.g., a name of the radiopharmaceutical), and the like from a laboratory technician.

Next, the nuclear medical imaging device 20 performs imaging based on the examination order information and generates a radiation image.

Next, the nuclear medical imaging device 20 generates and outputs a RRDSR (Radiopharmaceutical radiation dose Structured Report) specialized for the management of information on the amount of a drug administered in nuclear medicine, similarly to an RDSR (radiation dose Structured Report) used for the management of the exposure radiation dose in CT (Computed Tomography) or general X-ray imaging, on the basis of the generated radiographic image, together with information input by a laboratory technician. RRDSR Is information conforming to the DICOM standard, and is one of data formats representing radiation dose information. RRDSR Records patient information, examination information, and the like.

The nuclear medical imaging device 20 includes a storage section (storage) 21, and stores the generated RRDSR in the storage section 21.

The radiation dose management device 30 receives, for example, the examination order information from the RIS server 10, and associates the examination name included in the examination order information with the information on the diagnostic reference level corresponding to the examination. Next, the radiation dose management device 30 calculates the total of the actual administered amounts of the radiopharmaceuticals administered to the patient in the nuclear medical examination based on the RRDSR. Next, the radiation dose management device 30 determines whether or not the sum of the calculated actual administered amounts is greater than a reference value of a diagnostic reference level, that is, whether or not the administered amount is an excessive administered amount.

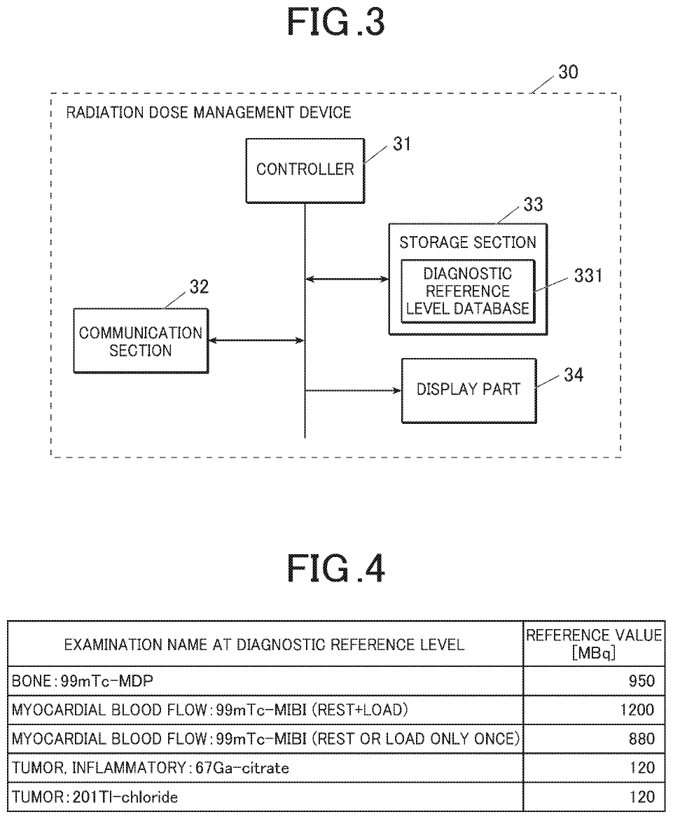

FIG. 3 shows a functional configuration of the radiation dose management device 30.

As shown in FIG. 3, the radiation dose management device 30 includes a controller 31, a communication section (communication interface) 32, a storage section (storage) 33, a display part (display) 34, and the like, which are connected to each other via a bus.

The controller 31 includes a central processing unit (CPU), a read only memory (ROM), a random access memory (RAM), and the like. The controller 31 comprehensively controls processing operation of each section of the radiation dose management device 30. Specifically, the CPU reads various processing programs stored in the ROM, develops the programs in the RAM, and performs various processes in cooperation with the programs.

The communication section 32 includes a network interface and the like. The communication section 32 transmits and receives data to and from external devices (the RIS server 10, the nuclear medical imaging device 20, and the like) connected through the communication network N. For example, the communication section 32 receives the examination order information from the RIS server 10 and receives the RRDSR from the nuclear medical imaging device 20.

The storage section 33 includes a hard disk drive (HDD), a nonvolatile memory, and the like, and stores various kinds of data.

The storage section 33 stores a diagnostic reference level database (DB) 331.

The diagnostic reference level database 331 are a database that stores diagnostic reference levels (DRLs) which are reference values for general radiation dose management in nuclear medicine for each examination name.

FIG. 4 illustrates an example of information stored in the diagnostic reference level database 331. As shown in FIG. 4, the diagnostic reference level database 331 stores reference values of radiation dose management in association with examination names in the diagnostic reference level.

Similarly to the examination name in the examination order information, the examination name in the diagnostic reference level includes information on the examination site, the type of the radiopharmaceutical (for example, the name of the radiopharmaceutical) administered to the patient in the examination, and the number of times of administration.

The examination site is, for example, a bone, myocardial blood flow, tumor, inflammation, tumor, or the like.

The names of the radiopharmaceuticals are, for example, 99 mTc-MDP, 99 mTc-MIBI, 67Ga-citrate, 201T1-chloride and the like.

As the number of times of administration, for example;(rest+load) indicates administration twice; and in the case of (rest or load only once) and in the case where there is no description about rest or load, it indicates administration once.

The display part 34 includes a monitor such as a liquid crystal display (LCD) or a cathode ray tube (CRT), and performs display in accordance with an instruction of a display signal input from the controller 31.

### (2 Operation of Radiation Dose Management System)

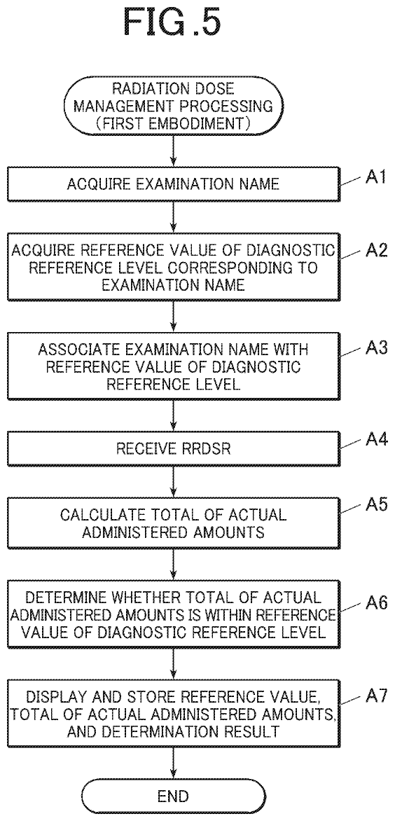

Next, the operation of the radiation dose management device 30 will be described. FIG. 5 is a flowchart illustrating the radiation dose management processing executed in the radiation dose management device 30. This processing is realized by software processing in cooperation between the CPU of the controller 31 and a program stored in the ROM.

The controller 31 receives the examination order information from the RIS server 10 and acquires the examination name included in the examination order information (step A1). That is, the controller 31 automatically acquires the examination name related to the examination of nuclear medicine from the storage section 11 (storage) of the RIS server 10 (radiation information device). The controller 31 functions as an examination information acquisition section (hardware processor). The step A1 is examination information acquiring. The examination name related to the examination of nuclear medicine is predetermined information related to the examination of nuclear medicine.

Next, the controller 31 acquires the reference value of the diagnostic reference level corresponding to the examination name acquired in step A1 from the diagnostic reference level database 331 (step A2).

For example, a case where the controller 31 acquires “bone scintigraphy (99mTc-MDP)” as the examination name in step A1 will be described. In this case, the controller 31 acquires “950 [MBq]” (refer to FIG. 4) as the reference value of the diagnostic reference level corresponding to the examination of “examination site: bone radiopharmaceutical name: 99 mTc-MDP number of times of administration: once”.

A case where the controller 31 has acquired “myocardial 99 mTc-MIBI (resting+load)” as the examination name in step Al will be described. In this case, the controller 31 acquires “1200 [MBq]” (see FIG. 4) as the reference value of the diagnostic reference level corresponding to an examination “examination site: myocardial blood flow radiopharmaceutical name: 99 mTc-MIBI administration frequency: twice”.

Next, the controller 31 associates the examination name acquired in step A1 with the reference value of the diagnostic reference level acquired in step A2 (step A3).

That is, the controller 31 automatically associates a examination name with information on a diagnostic reference level corresponding to the examination based on the examination name. The controller 31 functions as an associating section. The step A3 is an association process.

Next, the controller 31 receives the RRDSR output from the nuclear medical imaging device 20 (step A4).

Next, based on the RRDSR received in step A4, the controller 31 calculates the total of the actual administered amounts in one examination from the administered time, the administered amount, and the type of the radiopharmaceutical (for example, radiopharmaceutical name) of the radiopharmaceutical administered to the patient in one examination (step A5). The controller 31 specifies the examination to which the same examination ID is assigned as the same examination.

Since a radiopharmaceutical undergoes radioactive decay, its radioactivity decreases over time. The radiation dose management device 30 is connected to a pharmaceutical database (DB) (not shown) that stores data of radiopharmaceuticals. The pharmaceutical database stores data on the half-life of a radiopharmaceutical. The half-life is the time until the radioactivity of the radiopharmaceutical is reduced by half. The controller 31 obtains the amount of radioactivity of the radiopharmaceutical at the administration time from the administered amount and the half-life corresponding to the type of the radiopharmaceutical based on the reference time and the administration time. From this, the controller 31 calculates the actual administered amount of radiopharmaceutical. The reference time is a time serving as a reference for indicating how much radioactivity of the radiopharmaceutical of interest is at what point in time.

Next, the controller 31 determines whether or not the total actual administered amount calculated in step A5 is within the reference value of the diagnostic reference level associated in step A3. Next, the controller 31 determines that the administered amount is OK when the total of the actual administered amounts is within the reference value, and determines that the administered amount is NG (excessive administration) when the total of the actual administered amounts exceeds the reference value (step A6).

Next, the controller 31 displays the reference value of the diagnostic reference level associated in step A3, the total actual administered amount calculated in step A5, and the determination result in step A6 on the display part 34, and stores them in the storage section 33 (step A7). Next, the controller 31 ends the radiation dose management processing.

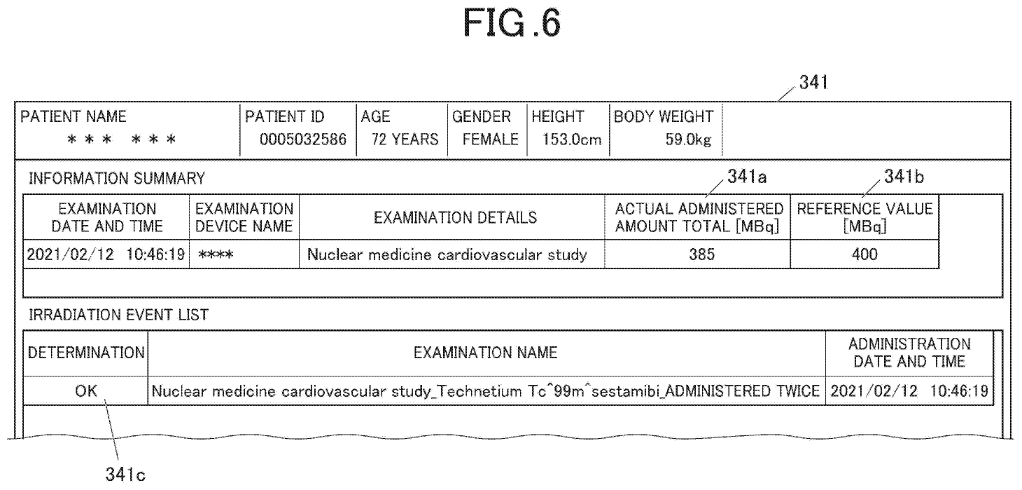

FIG. 6 shows an example of displaying the reference value of the diagnostic reference level, the total actual administered amount, and the determination result.

As shown in FIG. 6, the controller 31 displays a display screen 341 on the display part 34, and displays the total actual administered amount calculated in step A5 in the 341a of the field in the display screen 341. The controller 31 displays, in the field 341b, the reference value of the diagnostic reference level associated in step A3. The controller 31 displays the determination result in step A6 in the field 341c.

In the radiation dose management system 100 according to the present embodiment, the radiation dose management device 30 may be omitted and the RIS server 10 may function as a radiation dose management device. That is, the RIS server 10 functions as an examination information acquisition section and an associating section.

In this case, in step A1 of the radiation dose management processing, the RIS server 10 acquires, from the examination order information stored in the storage section 11, the examination name included in the examination order information. That is, the RIS server 10 automatically acquires the examination name related to the examination of the nuclear medicine from the storage section 11 (storage) of the RIS server 10 (radiation information device). The examination name related to the examination of nuclear medicine is predetermined information related to the examination of nuclear medicine.

The radiation dose management system 100 may include a hospital information system (HIS:Hospital Information Systems) (not shown) connected via the communication network N so as to be able to transmit and receive data. The hospital information system stores the examination order information in its storage section.

In this case, in step A1 of the radiation dose management processing, the examination information acquisition section (the controller 31 or the RIS server 10) acquires, from the examination order information received from the hospital information system, the name of the examination included in the examination order information. That is, the examination information acquisition section automatically acquires the examination name related to the examination of nuclear medicine from the storage section (storage) of the hospital information system. The examination name related to the examination of nuclear medicine is predetermined information related to the examination of nuclear medicine.

An external device outside the radiation dose management system 100 configured to be able to communicate with the radiation dose management device 30 may include a storage, and the storage may store the examination order information.

In this case, in step A1 of the radiation dose management processing, the examination information acquisition section (the controller 31) acquires, from the examination order information received from the external device, the examination name included in the examination order information. That is, the examination information acquisition section automatically acquires the examination name related to the examination of nuclear medicine from the storage of the external device. The examination name related to the examination of nuclear medicine is predetermined information related to the examination of nuclear medicine.

### (3. Technical Advantages)

As described above, the radiation dose management system 100 of the present embodiment includes the examination information acquisition section (the controller 31 or the RIS server 10) that automatically acquires predetermined information related to the examination of nuclear medicine from the storage. The radiation dose management system 100 according to the present embodiment includes the associating section (the controller 31 or the RIS server 10) that automatically associates predetermined information on an examination in nuclear medicine with information (reference value) on a diagnostic reference level corresponding to the examination.

The diagnostic reference level is a reference value for general radiation dose management in nuclear medicine. The examination name at the diagnostic reference level includes information on the site to be examined, the type of radiopharmaceutical administered to the patient in the examination (e.g., radiopharmaceutical name), and the number of times of administration.

Therefore, even in a case where a radiopharmaceutical is administered twice in one nuclear medical examination, an appropriate diagnostic reference level corresponding to the nuclear medical examination can be associated. Therefore, it is possible to more appropriately manage an exposure radiation dose of a patient in a nuclear medicine examination.

In a case where the predetermined information regarding the nuclear medicine examination is manually input to the radiation dose management device, since the predetermined information (for example, the examination name) regarding the nuclear medicine examination to be input is similar, there is a possibility that an input error occurs. However, since the examination information acquisition section in the radiation dose management system 100 of the present embodiment automatically acquires predetermined information related to the examination of nuclear medicine, it is possible to prevent such an input error.

In the radiation dose management system 100 of the present embodiment, the predetermined information related to the examination of the nuclear medicine is an examination name. The associating section associates the examination name with the information on the diagnostic reference level based on the examination name.

Therefore, by receiving the examination order information including the examination name from the RIS server 10 or the like, it is possible to easily acquire the predetermined information on the nuclear medical examination.

The radiation dose management system 100 according to the present embodiment includes a radiation information device (the RIS server 10). The radiation information device includes an examination information acquisition section. The examination information acquisition section acquires the examination name from the storage (the storage section 11, the storage section of the hospital information system, or the like).

Therefore, even when the radiation dose management system 100 does not include the radiation dose management device 30,

the exposure radiation dose of the patient can be more appropriately managed in the examination of nuclear medicine.

The radiation dose management system 100 according to this embodiment includes a radiation dose management device 30 and a radiation information device (RIS server 10) including a storage (storage section 11). The radiation dose management device 30 includes an examination information acquisition section (controller 31). The examination information acquisition section acquires the examination name from the storage (storage section 11) of the radiation information device.

Therefore, by receiving the examination order information including the examination name from the storage section 11 of the RIS server 10, it is possible to easily acquire the predetermined information on the nuclear medical examination.

### Second Embodiment

A description of a radiation dose management system 100 according to a second embodiment will be given next. The following descriptions will be made focusing on differences from the first embodiment.

In the present embodiment, the predetermined information related to a nuclear medicine examination is information on examinations included in one examination.

The information on the examination includes an examination site in a nuclear medical examination, the type of radiopharmaceutical (radiopharmaceutical name) administered to a patient, and the like.

The information on the examination is included in RRDSR output from the nuclear medical imaging device 20.

The radiation dose management device 30 according to the present embodiment receives the RRDSR from the nuclear medical imaging device 20 and associates the information on the RRDSR with the information on the diagnostic reference level corresponding to the examination. Next, the radiation dose management device 30 calculates the total of the actual administered amounts of the radiopharmaceuticals administered to the patient in the nuclear medical examination based on the RRDSR. Next, the radiation dose management device 30 determines whether or not the sum of the calculated actual administered amounts is greater than a reference value of a diagnostic reference level, that is, whether or not the administered amount is an excessive administered amount.

### (4. Operation Of Radiation Dose Management System)

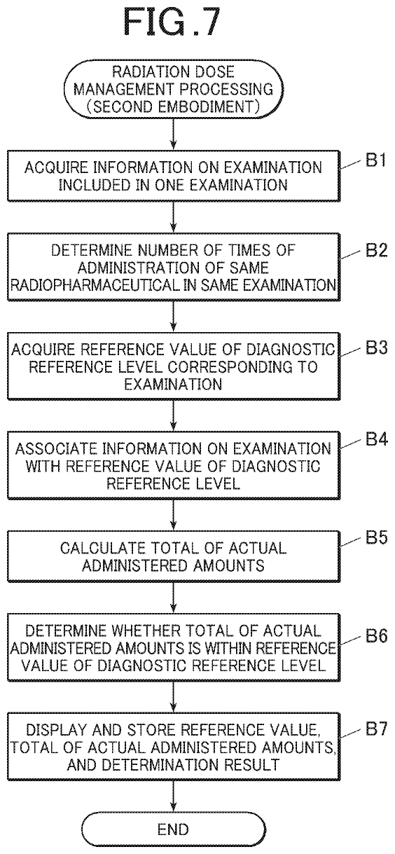

FIG. 7 shows a flowchart of a radiation dose management process in the radiation dose management system 100 of the present embodiment. This processing is realized by software processing in cooperation between the CPU of the controller 31 and a program stored in the ROM.

The controller 31 receives the RRDSR output from the nuclear medical imaging device 20 and acquires information on the examinations included in one examination in the RRDSR (step B1). That is, the controller 31 automatically acquires the information of the examinations included in one examination in the RRDSR related to the nuclear medical examination from the storage section 21 (storage) of the nuclear medical imaging device 20. The controller 31 functions as an examination information acquisition section. The step B1 is examination information acquiring. The information of examinations included in the one examination is predetermined information related to an examination of nuclear medicine.

The controller 31 specifies the examination to which the same examination ID is assigned as the same examination.

Next, based on the information on the examinations included in one examination acquired in step B1, the controller 31 determines the number of times of administration of the same radiopharmaceutical in the same examination (step B2).

Specifically, the controller 31 determines whether or not the information on the first radiopharmaceutical and the information on the second radiopharmaceutical in the information on the examinations included in one examination are the same. The controller 31 functions as a determination section.

For example, a case will be described in which the controller 31 has acquired, in step B1, “radiopharmaceutical name: 99 mTc-MDP (information on a first radiopharmaceutical)” and “radiopharmaceutical name: 99 mTc-MDP (information on a second radiopharmaceutical)” as information on examinations included in one examination. In this case, the controller 31 determines that the information of the first radiopharmaceutical and the information of the second radiopharmaceutical are the same. The controller 31 determines that the first radiopharmaceutical has been administered twice (a plurality of times) in one examination.

On the other hand, a case will be described in which the controller 31 has acquired, in step B1, “radiopharmaceutical name: 67Ga-citrate (information on a first radiopharmaceutical)” and “radiopharmaceutical name: 201T1-chloride (information on a second radiopharmaceutical)” as information on examinations included in one examination. In this case, the controller 31 determines that the information on the first radiopharmaceutical and the information on the second radiopharmaceutical are not the same. The controller 31 determines that each of the first radiopharmaceutical and the second radiopharmaceutical has been administered once in one examination.

Next, the controller 31 acquires the reference value of the diagnostic reference level corresponding to the information of the examination included in one examination acquired in step B1 from the diagnostic reference level database 331 (step B3).

For example, a case will be described in which the controller 31 has acquired, in step B 1, “examination site: myocardial blood flow radiopharmaceutical name: 99 mTc-MIBI (information on a first radiopharmaceutical)” and “examination site: myocardial blood flow radiopharmaceutical name: 99 mTc-MIBI (information on a second radiopharmaceutical)” as information on examinations included in one examination. In this case, the controller 31 determines that the first radiopharmaceutical has been administered twice in one examination in step B2. Next, in Step B3, the controller 31 acquires “1200 [MBq]” (see FIG. 4) as the reference value of the diagnostic reference level corresponding to the examination of “examination site: myocardial blood flow radiopharmaceutical name: 99 mTc-MIBI number of times of administrations: twice”.

On the other hand, a case will be described in which the controller 31 has acquired “examination site: tumor/inflammation radiopharmaceutical name: 67Ga-citrate (information on the first radiopharmaceutical)” and “examination site: tumor radiopharmaceutical name: 201T1-chloride (information on the second radiopharmaceutical)” as the information on the examinations included in the one examination in step B1. In this case, the controller 31 determines in step B2 that each of the first radiopharmaceutical and the second radiopharmaceutical has been administered once in one examination. Next, in step B3, the controller 31 acquires “120 [MBq]” (see FIG. 4) as the reference value of the diagnostic reference level corresponding to the examination “examination site: tumor/inflammation radiopharmaceutical name: 67Ga-citrate number of times of administration: once”. The controller 31 acquires “120 [MBq] ” (see FIG. 4) as the reference value of the diagnostic reference level corresponding to the examination of “examination site: tumor radiopharmaceutical name: 201T1-chloride number of times of administration: once”.

Next, the controller 31 associates the information on the examinations included in one examination acquired in step B1 with the reference value of the diagnostic reference level acquired in step B3 (step B4).

That is, the controller 31 associates the information on the examination included in one examination with the reference value of the diagnostic reference level (information on the diagnostic reference level) based on the determination result by the determination section. The controller 31 functions as an associating section. The step B4 is associating.

A case where the controller 31 serves as the determination section to determine that the first radiopharmaceutical has been administered a plurality of times will be described. In this case, the controller 31 associates the information on the examination included in one examination with the information on the diagnostic reference level on the basis of the determination result indicating that the first radiopharmaceutical has been administered plural times.

Next, the controller 31 performs steps B5 to B7 similar to steps A5 to A7 of the radiation dose management processing of the first embodiment, and ends the radiation dose management processing.

### (5. Technical Advantages)

As described above, in the radiation dose management system 100 according to the present embodiment, the predetermined information related to the examination of the nuclear medicine is the information of the examination included in one examination. The radiation dose management system 100 according to the present embodiment includes the determination section (the controller 31) that determines whether or not the information on the first radiopharmaceutical and the information on the second radiopharmaceutical included in one examination are the same. The associating section (controller 31) associates the information on the examinations included in one examination with the information on the diagnostic reference level based on the determination result by the determination section.

Therefore, even in a case where a radiopharmaceutical is administered twice in one nuclear medical examination, an appropriate diagnostic reference level corresponding to the nuclear medical examination can be associated. Therefore, it is possible to more appropriately manage an exposure radiation dose of a patient in a nuclear medicine examination.

In the radiation dose management system 100 of the present embodiment, when determining that the information on the first radiopharmaceutical and the information on the second radiopharmaceutical are the same, the determination section determines that the first radiopharmaceutical has been administered a plurality of times. The associating section associates the information on the examinations included in one examination with the information on the diagnostic reference level based on a determination result that the first radiopharmaceutical has been administered a plurality of times by the determination section.

Therefore, even when a radiopharmaceutical is administered a plurality of times in one nuclear medical examination, an appropriate diagnostic reference level corresponding to the nuclear medical examination can be associated. Therefore, it is possible to more appropriately manage an exposure radiation dose of a patient in a nuclear medicine examination.

The radiation dose management system 100 according to this embodiment includes a radiation dose management device 30 and a nuclear medical imaging device 20 including a storage (storage section 21). The radiation dose management device 30 includes an examination information acquisition section (controller 31). The examination information acquisition section acquires predetermined information related to an examination of nuclear medicine from the storage (storage section 21) of the nuclear medical imaging device 20.

Therefore, by receiving the RRDSR from the nuclear medical imaging device 20, it is possible to easily acquire the predetermined information on the nuclear medical examination.

In the radiation dose management system 100 of the present embodiment, the predetermined information related to the nuclear medical examination is information of a radiopharmaceutical radiation dose structured report (RRDSR).

Therefore, when the examination information acquisition section acquires the predetermined information related to the nuclear medical examination from the storage section 21 of the nuclear medical imaging device 20, communication according to the standard of the RRDSR can be performed. Therefore, when the examination information acquisition section acquires the predetermined information, it is not necessary to check whether information can be actually communicated between the device that transmits the predetermined information and the radiation dose management device 30. The cost for confirming whether communication can be performed between the device that transmits the predetermined information and the radiation dose management device 30 is also unnecessary. It is possible to save time and effort for manually inputting the predetermined information related to the nuclear medical examination in the radiation dose management device 30.

The description in the above embodiment is an example of the radiation dose management system according to the present invention, and the present invention is not limited to this.

For example, in the above-described embodiment, a case where a radiopharmaceutical is administered twice in one nuclear medical examination and a case where a radiopharmaceutical is administered only once in one nuclear medical examination have been described. However, the number of times of administration of the radiopharmaceutical in one nuclear medical examination is not limited thereto.

For example, although the example in which a semiconductor memory or an HDD is used as a computer-readable medium storing a program for executing each processing has been disclosed in the above embodiment, it is not limited to this example. As other computer-readable media, portable recording media such as CD-ROMs can also be applied. As a medium for providing program data via a communication line, a carrier wave may be used.

In addition, the detailed configuration and the detailed operation of each section constituting the radiation dose management system can be appropriately changed without departing from the scope of the present invention.

As for the other detailed configurations and the detailed operations of devices forming the analysis processing system, modifications can be made as needed within the scope of the present invention.

Although the disclosure has been described with respect to only a limited number of embodiments, those skilled in the art, having benefit of this disclosure, will appreciate that various other embodiments may be devised without departing from the scope of the present invention. Accordingly, the scope of the invention should be limited only by the attached claims.

What is claimed is:

1. A radiation dose management system comprising:
a hardware processor that:
automatically acquires, from a storage, predetermined information related to an examination of nuclear medicine, and
automatically associates the predetermined information with information on a diagnostic reference level corresponding to the examination.

2. The radiation dose management system according to claim 1, wherein
the predetermined information is an examination name, and
the hardware processor associates the examination name with the information on the diagnostic reference level based on the examination name.

3. The radiation dose management system according to claim 2, further comprising:
a radiation information device that comprises the hardware processor, wherein
the hardware processor acquires the examination name from the storage.

4. The radiation dose management system according to claim 2, further comprising:
a radiation dose management device that comprises the hardware processor; and
a radiation information device that comprises the storage, wherein
the hardware processor acquires the examination name from the storage of the radiation information device.

5. The radiation dose management system according to claim 1, wherein
the predetermined information is information on an examination included in one examination,
the one examination includes, as the information on the examination, information on a first radiopharmaceutical and information on a second radiopharmaceutical, and
the hardware processor:
determines whether the information on the first radiopharmaceutical and the information on the second radiopharmaceutical are same, and
associates the information on the examination with the information on the diagnostic reference level based on a determination result.

6. The radiation dose management system according to claim 5, wherein
the hardware processor:
upon determining that the information on the first radiopharmaceutical and the information on the second radiopharmaceutical are the same, determines that the first radiopharmaceutical has been administered a plurality of times, and
associates the information on the examination with the information on the diagnostic reference level based on a determination result that the first radiopharmaceutical has been administered the plurality of times.

7. The radiation dose management system according to claim 5, comprising:
a radiation dose management device that comprises the hardware processor; and
a nuclear medical imaging device that comprises the storage, wherein
the hardware processor acquires the predetermined information from the storage of the nuclear medical imaging device.

8. The radiation dose management system according to claim 7, wherein the predetermined information is information on a Radiopharmaceutical Radiation Dose Structured Report (RRDSR).

9. A radiation dose management device comprising:
a hardware processor that:
automatically acquires, from a storage, predetermined information related to an examination of nuclear medicine, and
automatically associates the predetermined information with information on a diagnostic reference level corresponding to the examination.

10. A radiation dose management method comprising:
automatically acquiring, from a storage, predetermined information related to an examination of nuclear medicine; and
automatically associating the predetermined information with information on a diagnostic reference level corresponding to the examination.

11. A non-transitory recording medium storing a computer readable program causing a computer of a radiation dose management system to:
function as a hardware processor that:
automatically acquires, from a storage, predetermined information related to an examination of nuclear medicine, and
automatically associates the predetermined information with information on a diagnostic reference level corresponding to the examination.

12. The recording medium according to claim 11, wherein
the predetermined information is an examination name, and
the hardware processor associates the examination name with the information on the diagnostic reference level based on the examination name.

13. The recording medium according to claim 12, wherein
the radiation dose management system comprises a radiation information device that comprises the computer functioning as the hardware processor, and
the hardware processor acquires the examination name from the storage.

14. The recording medium according to claim 12, wherein
the radiation dose management system comprises:
a radiation dose management device that comprises the computer functioning as the hardware processor; and
a radiation information device that comprises the storage,
the hardware processor acquires the examination name from the storage of the radiation information device.

15. The recording medium according to claim 11, wherein
the predetermined information is information on an examination included in one examination,
the one examination includes, as the information on the examination, information on a first radiopharmaceutical and information on a second radiopharmaceutical, and
the hardware processor:
determines whether the information on the first radiopharmaceutical and the information on the second radiopharmaceutical are same, and
associates the information on the examination with the information on the diagnostic reference level based on a determination result.

16. The recording medium according to claim 15, wherein
the hardware processor:
upon determining that the information on the first radiopharmaceutical and the information on the second radiopharmaceutical are the same, determines that the first radiopharmaceutical has been administered a plurality of times, and
associates the information on the examination with the information on the diagnostic reference level based on a determination result that the first radiopharmaceutical has been administered the plurality of times.

17. The recording medium according to claim 15, wherein
the radiation dose management system comprises:
a radiation dose management device that comprises the computer functioning as the hardware processor; and
a nuclear medical imaging device that comprises the storage,
the hardware processor acquires the predetermined information from the storage of the nuclear medical imaging device.

18. The recording medium according to claim 17, wherein the predetermined information is information on a Radiopharmaceutical Radiation Dose Structured Report (RRDSR).
